(19) United States
(12) Patent Application Publication
(10) Pub. No.: US 2013/0245444 A1
(43) Pub. Date: Sep. 19, 2013

(54) SYNCHRONOUSLY PUMPED ULTRASONIC WAVES AND SHEAR WAVE GENERATION BY SAME

(71) Applicant: Andrey Rybyanets, Rostov on Don (RU)

(72) Inventor: Andrey Rybyanets, Rostov on Don (RU)

(73) Assignee: WAVOMED LTD., Ramat Gan (IL)

(21) Appl. No.: 13/825,327

(22) PCT Filed: Sep. 20, 2011
(86) PCT No.: PCT/IB2011/054114

§ 371 Date: Mar. 21, 2013

Related U.S. Application Data
(60) Provisional application No. 61/385,163, filed on Sep. 22, 2010.

(51) Int. Cl. A61B 8/00

(52) U.S. Cl. CPC A61B 8/00
USPC 600/439

(57) ABSTRACT

Methods and apparatus for producing synchronously pumped (SP) ultrasonic bursts which can be used to create ultrasound radiation pressure. The emission of an ultrasonic burst by a piezoelement is synchronized with the reflection of another ultrasonic burst by the same piezoelement to create a combined ultrasonic burst with increased amplitude. Repeated synchronized emissions and reflections of ultrasonic bursts lead to resonant growth of a burst amplitude to equal that of a standing wave, but without formation of a nodal structure. In some embodiments, the SP ultrasonic bursts generate shear waves. In some embodiments, the shear waves are resonant shear waves. In some embodiments, the shear waves are formed in a supersonic regime. Shear waves thus formed can be used for various treatments of biological tissues, with or without RF heating.

### CROSS REFERENCE TO RELATED APPLICATIONS

This application claims priority from U.S. Provisional Patent Application No. 61/385,163 filed Sep. 22, 2010, which is incorporated herein by reference in its entirety.

### FIELD AND BACKGROUND

Embodiments of the invention disclosed hereinbelow relate in general to ultrasound apparatus amd methods and in particular to generation of ultrasonic bursts and their use in various media.

Various methods are known for delivering and coupling acoustic (also referred to herein as “ultrasound” or “ultrasonic”) energy to a region of tissue to perform a diagnostic and/or therapeutic and/or cosmetic procedure on a patient's tissue. Among such procedures are, for example, non-invasive assaying of blood analytes, drug delivery by phonophoresis, lithotripsy, tissue ablation, and lysis of fat cells for cosmetic removal of adipose tissue.

For many types of therapeutic and/or cosmetic acoustic applications, such as for example lithotripsy, tissue ablation and lysis noted above, sufficient acoustic energy must be delivered to a tissue region to destroy and remove tissue in the region. Generally, the acoustic energy is delivered by focusing at least one beam of relatively intense ultrasound on the region. The high intensity, focused ultrasound, conventionally referred to by the acronym “HIFU”, may be used to generate various thermal and mechanical effects on tissue that include local heating of tissue and/or cavitation that disrupts and destroys the tissue. Tissue raised to and maintained at a temperature above about 42° C. dies rapidly, and mechanical stresses generated by cavitation breach and tear cell membranes of the tissue.

However, it is often difficult to efficiently treat relatively large volumes of tissue using HIFU. For example, HIFU beams are often focused to relatively small volumes of tissue and can require relatively large dwell times at the focal volumes to destroy tissue therein. Typically, a focal volume of a HIFU beam is substantially contained within a prolate ellipsoid. For a frequency of ultrasound equal to about 200 kHz, which is commonly used in ultrasound tissue treatment, the ellipsoid has a long axis of about 15 mm along a direction of propagation of the beam and a maximum cross section perpendicular to the propagation direction having a diameter of about 7.5 mm. For frequency of about 1 MHz, the long axis is about 3 mm and the cross section diameter is about 1.5 mm. In general, the focal volume has a lateral diameter of approximately 1 wavelength and a length of between about 2-3 wavelengths. Boundaries of the focal volume are assumed to be in regions where acoustic intensity is attenuated by about 6 dB. Treating an extended region of tissue with HIFU, for example to lyse adipose tissue, can therefore often be a relatively tedious task that requires a relatively long time to perform. As a result, various techniques have been proposed and/or used for expanding a useful focal volume of HIFU beams and for electronically and/or mechanically scanning the beams to treat relatively large tissue volumes.

However, controlling HIFU beams to deliver effective acoustic energy which is spatially relatively homogenous over an extended tissue volume that is a desired target for treatment and which does not adversely affect non-target tissue can be problematic. Often configurations of extended focal volume HIFU beams exhibit “hot spots” that limit therapeutic and/or cosmetic use of the beams. Also, ultrasound propagated into the body so that it is substantially focused in a desired region, generally propagates through and past the focal region and is incident on organs and/or body features for which the ultrasound is not intended.

For example, adipose tissue generally resides in the subcutaneous layer of the skin and is located in a region from about a few mm to a few tens of mm below the skin surface. In procedures for tissue ablation and lysis of fat cells for cosmetic removal of adipose tissue, ultrasound focused to fat tissue below the skin may propagate beyond the adipose tissue, and impinge on and damage internal organs and body features lying below the subcutaneous layer. If the ultrasound is being used to treat belly fat, the ultrasound may, for example, be incident on the liver. If the ultrasound is used to treat cellulites in the hip region, the ultrasound may be incident on and reflected from bone tissue below the skin. The reflected ultrasound can interfere with the ultrasound propagated into the body to treat the cellulites and generate a standing acoustic wave having intensity at or near the skin surface that can damage the skin.

FIG. 1a shows schematically a cross section of a perspective view of an ultrasound treatment apparatus 50 (UTA) as disclosed in PCT/IB2010/052624. UTA 50 comprises a vacuum vessel 52 for drawing up and holding a region of tissue region 40 to be treated with ultrasound, and an array of ultrasound transducers 30 for generating the ultrasound. In an embodiment, transducers 30 are interfaced to tissue region 40 by protective buffer layers 132. Buffer layers 132 have thickness equal to about a quarter wavelength of ultrasound generated by transducers 30 and acoustic impedance substantially equal to about that of tissue 40. Optionally, vacuum vessel 52 may have other shapes and/or the transducers may be arranged in different arrangements. UTA 50 comprises a “shape adapter” that distorts the shape of tissue drawn up into vacuum vessel 52 so that the drawn up tissue makes substantially continuous and intimate contact with buffers 132. In an embodiment, the shape adapter comprises a “plunger” 60 having a stem 62 that protrudes into vacuum vessel 52 through an aperture 53 and may be mounted with a plunger head 64. Optionally, stem 62 comprises a pipe 66 formed having spiracles 67 through which air may be aspirated from vessel 52 to provide partial vacuum for drawing skin into the vessel. The pipe may be sealed in aperture 53 to reduce, leakage of air between the pipe and walls of aperture so that a suitable partial vacuum for drawing up skin can be created in vessel 52. Arrows 68 schematically represent flow of air aspirated from vessel 52. Optionally, plunger head 64 may be an oblate, “pumpkin shaped”, body. In some embodiments, the position of plunger 60 may be fixed and located so that when tissue 40 is drawn up into vessel 52, plunger head 64 deforms the drawn up tissue, as shown in FIG. 1, so that it spreads laterally and makes close, continuous contact with buffers 132.

In some embodiments, plunger 60 may be configured so that it is moveable “up” and “down” to adjust depth to which plunger head 64 intrudes into vessel 52. Optionally, plunger head 64 may be moved up and down by moving stem 62 along its length in aperture 53. Sealing of stem 62 in aperture 53 to maintain suitable reduced leakage of air between the stem and aperture may be provided using any of various methods and materials known in the art. For example, stem 62 might be sealed against air leaks using a configuration of o-rings and/or vacuum greases.

It was noted that shape adapters may be different from plunger 60, having for example a cylindrical or annular shaped plunger head. Alternatively, a plunger head may comprise a plurality of component elements such as an array of parallel cylinders that are pressed to skin drawn into vacuum vessel 52. Plunger head 64 may be formed from an elastic membrane and displacement of the plunger head may be accomplished by filling the plunger head with a gas or liquid to expand it, or by removing gas or liquid to cause the plunger head to contract.

As described in PCT/IB2010/052624, plunger head 64 is repetitively moved during illumination of tissue region 40 with ultrasound to change a force applied by the plunger head on the tissue region and thereby change the position of mass points in the region relative to ultrasound transmitted by transducers 30. To change the position of mass points, vacuum vessel 52 may be perturbed at a suitable frequency. The spatial shifting of mass points in tissue region 40 aids in homogenizing the effects of the ultrasound in the tissue region. Optionally, repetitive motion of plunger head 64 may be performed at a frequency substantially equal to a mechanical relaxation time and/or resonant frequency of tissue region 40. Mechanical resonant and relaxation time frequencies of skin tissue range from about 300 Hz to about 10 kHz. By mechanically vibrating tissue region 40 at a resonant frequency, motion of mass points in the tissue region tend to be relatively large and effects such as tissue heating by the motion are amplified. By mechanically vibrating tissue region 40 at a relaxation time frequency, cavitation effects in the tissue region are also amplified.

Ultrasonic shear waves are used in non-destructive testing (NDT) and diagnostics of different materials. The use of an acoustic radiation force to remotely generate low-frequency shear waves in viscoelastic media (tissue, rubber like media, etc) is also known. Shear waves generation by pulsed radiation force created by HIFU has been proposed for diagnostic and imaging methods in tissue (see e.g. Bercoff J.; et al. Ultrasound Medicine and Biology. 2003, vol. 13, pp. 143-152 and Bercoff J. ey al., IEEE Transactions on Ultrasonics, Ferroelectrics, and Frequency Control. 2004, vol. 51, pp. 396-409). However, shear waves cannot propagate via fluids that prohibit use of immersion methods.

The main problems in using shear waves remotely generated by HIFU for NDT purposes (elasticity estimation, shear modulus measurements, etc.) relate to difficulties of introducing and focusing HIFU in rubber like media, as well as to providing acoustic contact between a HIFU transducer and tested objects. There is therefore a need for, and it would be advantageous to have, apparatus and methods of producing ultrasound radiation pressure and shear waves in a more efficient way for various NDT and tissue treatment purposes.

### SUMMARY

Embodiments disclosed herein provide methods and apparatus for producing Synchronously Pumped (SP) ultrasonic waves which can be used to create ultrasound radiation pressure. Synchronous pumping is obtained by synchronizing the emission, by a piezoelement, of an ultrasonic burst wave (also referred to simply as “ultrasonic burst” or just “burst”) with opposite polarity to a burst reflected by the same piezoelement, such that the opposite polarity-emitted and reflected bursts combine to form a burst with increased amplitude. Repeats of this action lead to resonance growth of the burst amplitude to equal that of a standing wave but without formation of a nodal structure.

Some embodiments disclosed herein provide methods and apparatus for producing shear waves in a SP regime. Other embodiments provide methods and apparatus for producing shear wave resonance (SWR) in a SP regime. Yet other embodiments provide methods and apparatus for producing supersonic shear waves (SSW) in a SP regime. Yet other embodiments provide apparatus which include radio frequency (RF) electrodes.

SP ultrasonic waves as disclosed herein may be used to create ultrasonic pressure in lossy viscoelastic media (e.g. polymers or resins) or in solids having high attenuation (e.g. composites). Methods and apparatus disclosed herein can be used for non-destructive testing (NDT) or diagnostics (e.g. to measure shear elasticity) in media mentioned above, i.e. in polymers, resins or composites). Alternatively, methods and apparatus disclosed herein can be be used in biological media (e.g. biological tissue) for example for treating relatively large regions of tissue with SP ultrasonic waves. In apparatus also including RF electrodes, methods for providing SP and SWR or SSW in the SP regime may also provide RF heating for “combined treatements” of biological tissue. Such combined treatments may include lypolytic, therapeutic and/or cosmetic treatments of tissue.

### BRIEF DESCRIPTION OF THE DRAWINGS

Non-limiting examples of embodiments disclosed herein are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1a shows schematically a cross section of a perspective view of an ultrasound treatment apparatus (UTA) as disclosed in PCT/IB2010/052624;

FIG. 1b shows a cylindrical piezoelement C;

FIG. 1c shows a two planar piezoelement transducer configuration;

FIG. 2a shows schematically a burst of a “direct” ultrasonic wave generated by a first transducer T1 or by cylindrical element C, which propagates forward and creates a radiation force F_{rad};

FIG. 2b shows schematically the burst in FIG. 2a after reflection from a solid boundary (a second transducer T2 or C itself) and amplification by the burst emitted by

T2 in opposite polarity;

FIG. 3a shows schematically the superposition of direct and reflected bursts at reflection from a cylindrical or a planar piezoelement (without pumping);

FIG. 3b shows schematically the superposition of direct and amplified (double amplitude) reflected bursts in a SP regime;

FIG. 4a shows schematically a cross section of a perspective view of an embodiment of an apparatus for treatments using a combination of SP and RF;

FIG. 4b shows in detail (side and upper view) an RF metal electrode mounted to a plunger in the apparatus of FIG. 4a;

FIG. 5a shows schematically a cross section of a perspective view of an apparatus according to another embodiment disclosed herein, having a shear mode receiving piezoelement mounted to a plunger;

FIG. 5b shows details of a shear mode receiving piezoelement mounted to a plunger;

FIG. 5c shows a combination of an RF electrode and a shear mode receiving piezoelement mounted on a plunger head;

FIG. 6 shows schematically the generation of shear waves by an ultrasonic burst;

FIG. 7 shows schematically the formation of shear waves from the interaction of the direct and reflected bursts in a SP regime;

FIG. 8 shows schematically one use of an apparatus with multi-electrode parallel or cylindrical transducers to produce shear waves;

FIG. 9 shows schematically another use of the apparatus of FIG. 8 to produce shear waves;

FIG. 10 shows schematically one use of an apparatus with multi-electrode transducers to produce supersonic shear waves;

FIG. 11 shows schematically another use of an apparatus with multi-electrode transducers to produce supersonic shear waves;

FIG. 12 shows schematically a physical mechanism of combinational treatment using SP and RF.

### DETAILED DESCRIPTION

### Synchronous Pumping (SP)

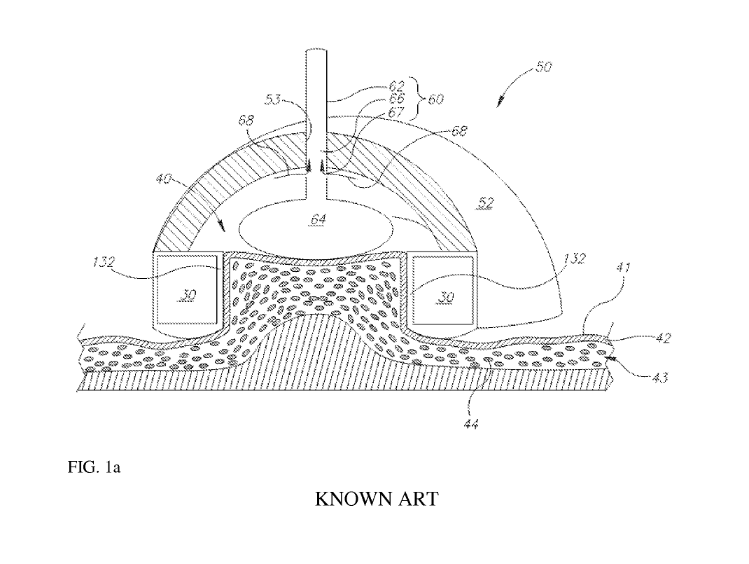

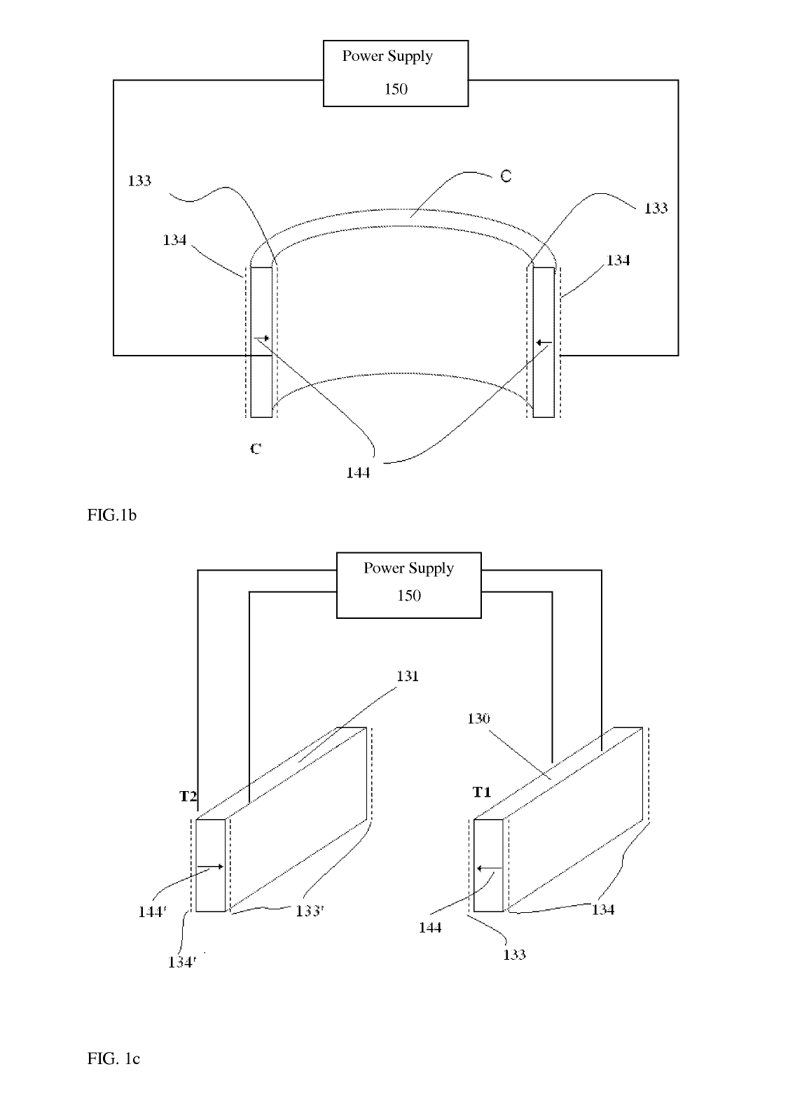

The present inventor has determined that an apparatus similar to apparatus 50 in FIG. 1a can be used for “Synchronous Pumping” (SP) of ultrasonic burst waves. SP is defined hereinbelow. While SP of ultrasonic burst waves is described next in detail with reference to a biological tissue medium, it is to be understood that apparatus and methods disclosed herein may equally be used with other media. For example, apparatus and methods disclosed herein may be used for SP of ultrasonic waves in lossy viscoelastic media (e.g. polymers or resins) or in solids having high attenuation (e.g. composites) and for non-destructive testing (NDT) or diagnostics in such media.

In an embodiment shown in FIG. 1b, a transducer similar to transducer 30 is in the shape of a cylindrical piezoelement C, which includes a front electrode 133 and a back electrode 134 which yield a piezoelement polarization 144. In another embodiment shown in FIG. 1c, transducers similar to transducer 30 are organized as two planar and parallel transducers T1 and T2. T1 includes a first planar piezoelement 130 and a front electrode 133 and a back electrode 134 which yield a piezoelement polarization 144. T2 includes a second planar piezoelement 131 and a front electrode 133′ and a back electrode 134′ which yield a piezoelement polarization 144′. In both embodiments, the transducers are coupled to a power supply power supply 150 which includes a frequency generator (not shown) and a controller (not shown), and which is configured to provide ultrasonic bursts with appropriate frequency, bursts parameters and phase shifts. In particular, the power supply is configured to excite each piezoelement to emit a direct ultrasonic burst in synchronization with the reflection of a previously emitted ultrasonic burst arriving at the same piezoelement, whereby the direct ultrasonic burst and the reflected ultrasonic burst combine to form a resonantly amplified ultrasonic burst wave in a SP regime (see below).

If short bursts are supplied on piezoelement C (or on opposite piezoelements T1 and T2) and if the burst duration is shorter than a transit time between elements, then a standing wave will not form. It is well known that an ultrasonic wave is reflected from a rigid boundary (piezoelement) in an opposite phase (180°). Therefore, an ultrasonic burst excited by a cylindrical piezoelement arrives at the (diametrically opposite side of the cylindrical) piezoelement and is reflected by it with a change of 180° in phase. The present inventor has determined that if the cylindrical piezoelement is switched on in opposite polarity with a delay equal to a transit time T of a previous burst each time it receives (reflects) a previous burst (“synchronization”), there will be a resonant growth of the burst amplitude equal to that of a standing wave, but without formation of the nodal structure typical for standing waves. A similar effect of resonant growth of the burst amplitude can be created using the T1 and T2 planar configuration. This effect (and the action causing it) are named herein “Synchronous Pumping”. An SP wave has a running wave character.

Let D be the diameter of cylindrical piezoelement C or the distance between T1 and T2. Let the frequency of the burst be f, its amplitude be A, and the propagation velocity in tissue be V. The transit time “T” of an ultrasonic wave between T1 and T2 or between opposite sides of a cylinder is then T=D/V.

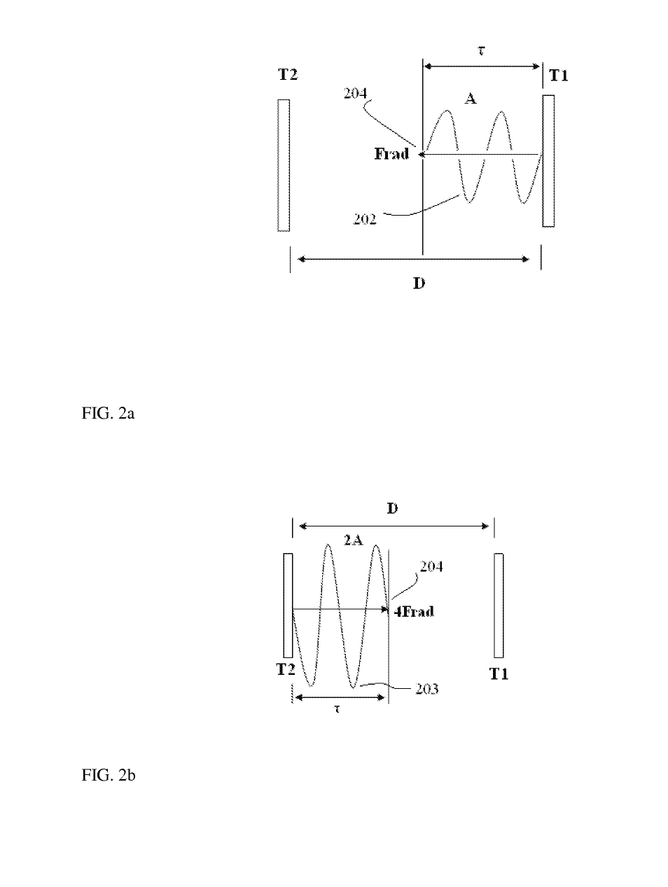

Let C or T1 be excited by a first electrical burst signal with a burst length in tissue=τ and with a frequency f, and let τ=T/2 (see FIG. 2a). In a general case, to prevent formation of standing waves as a result of the superposition of direct and reflected waves, T must be less then T and preferably less than T/2. The excitation provides a first burst 202 which propagates to the left in FIG. 2a. While propagating, the burst produces a radiation force F_{rad} 204. At time t₁=T, burst 202 arrives at a diametrically opposite (“second”) side of C or at T2 and is reflected therefrom. At this moment, C (or T2) is excited by an identical electrical burst signal with a burst length τ=T/2, frequency f and amplitude A, but with opposite polarity, to produce a second burst identical with the first and in phase with the reflected burst (see FIG. 2b). The reflected burst thus acquires an added amplitude A, becoming an “amplified” burst 203 propagating from left to right and having a 2A amplitude. At time t2=T, amplified burst 203 arrives at the first side of C (or at T1) and is reflected therefrom. At this moment, C (or T1) emits again a (third) burst identical (in phase and polarity) with the first burst. The third burst is in phase with, and added in amplitude to the reflected burst, which now has 3A amplitude and propagates from right to left (not shown). In terms of timing, C (or T1) generates a burst with length τ at times t=0, 4τ, 8τ etc. (and more generally, at t=4NτT, where N=0, 1, 2, 3 . . . etc.). Then C (or T2) generates a burst with length τ at times t=2τ, 6τ, 10τ etc (and more generally, at t=2+4Nτ, where N=0, 1, 2, 3 . . . etc.). In principle, this process can continue to infinity, resulting in ultrasonic burst resonance amplification (restricted only by ultrasonic wave attenuation).

Note that while the excitations described in the embodiment above were done in sequence, first on one piezoelement then on the other, in other embodiments they can also be done not in sequence, for example simultaneously.

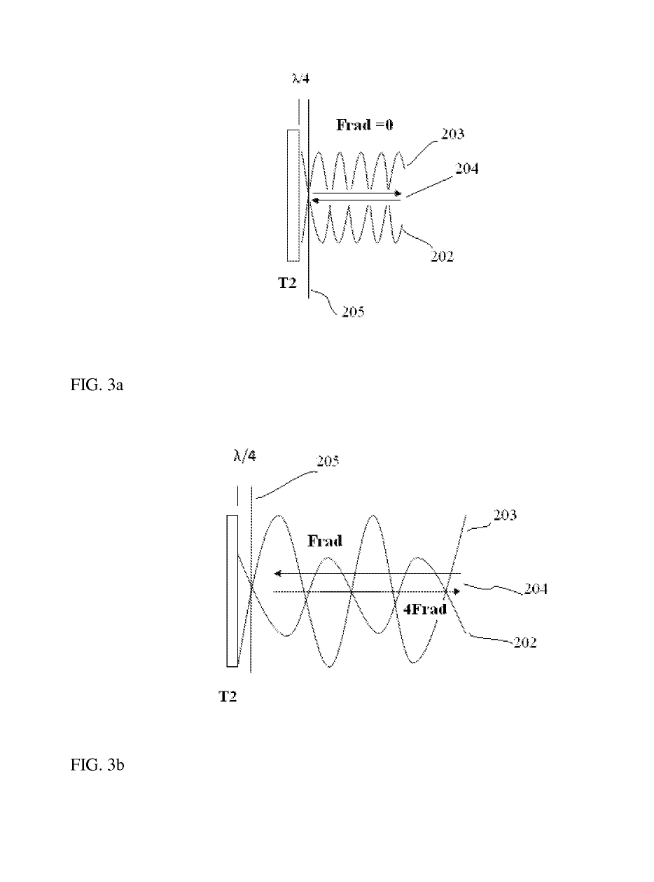

In synchronous pumping, upon (during) burst reflection, direct and reflected waves have opposite polarity, as in the case of standing waves. As shown in FIG. 3a, in the standing wave case the waves compensate each other at odd λn/4 distances (n=1, 3, 5, 7 . . . etc), with a first compensation “plane” at distance λ/4 marked 205. In contrast, the compensation process using bursts is transient, and in SP there is compensation only during burst reflection (“transient” compensation). That is, in the standing wave case, if the skin is positioned at the λ/4 plane there is a node of pressure on the skin at all times. In contrast, in burst mode there is zero pressure on the skin at that position only during the superposition of direct and reflected bursts. Further in contrast, at t1 (beginning of the first reflection process) the amplitude of the reflected and amplified burst is 2A, while the direct (received) burst has an amplitude A. Therefore, as shown in FIG. 3b, in SP the compensation is not full and instead of zero pressure at λ/4 distance from the boundary (as in standing waves), there is a pressure with amplitude A after each instance of reflection. Note that if one accounts for acoustic attenuation, the burst amplitude grows to and saturates at Q×A, where Q is a mechanical quality factor.

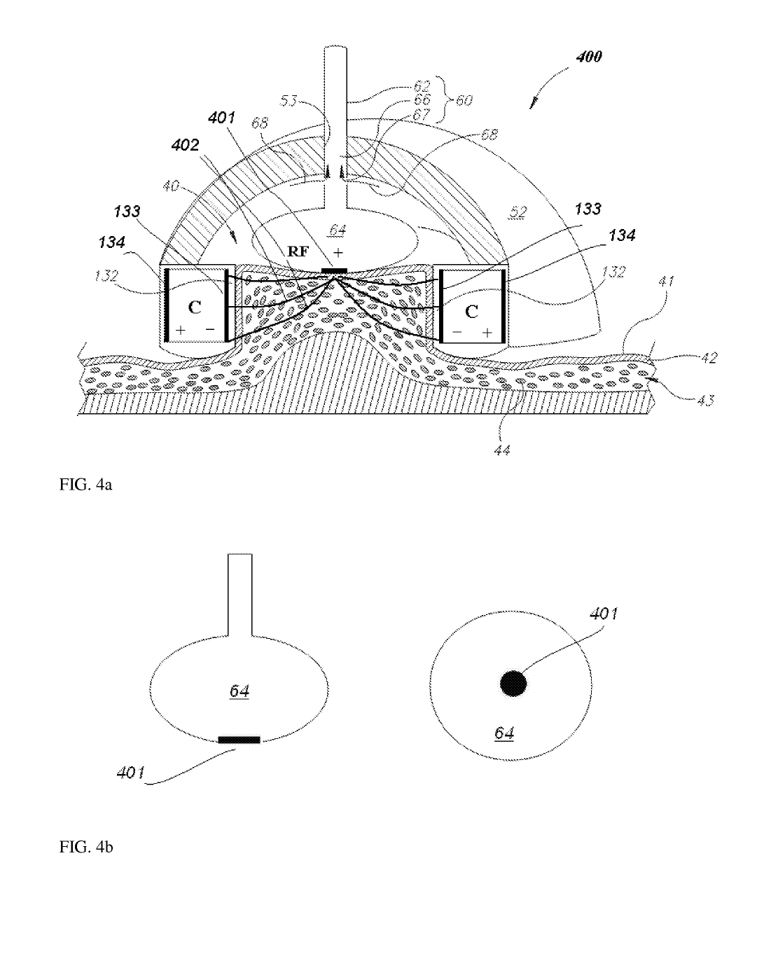

FIG. 4a shows schematically a cross section of a perspective view of an apparatus 400 for treatments using a combination of SP and RF according to an embodiment disclosed herein. Apparatus 400 includes, in addition to elements shown in apparatus 50, a plunger electrode 401 attached to plunger head 64. Plunger electrode 401 may be a simple metal disk which has exemplarily a cylindrical shape (FIG. 4b), with a typical diameter of a few mm. In general, electrode 401 may be made of any biocompatible, electrically conductive material (e.g. Cr, Au, stainless steel, etc) and can be electrically connected to a power source by wires (not shown). Apparatus 400 further includes a cylindrical piezoelement C with electrodes 133 and 134. Electrode 401 is commonly connected with back electrode 134 to an AC source and used in conjunction with front electrode 133 to generate an RF electric field indicated by field lines 402 by applying opposite polarities to these two electrodes. Exemplarily, a polarity “+” is applied to electrode 401, while a polarity “−” is applied to electrode 133. Alternatively, “−” is applied to electrode 401 and “+” is applied to electrode 133. In other embodiments, C may be replaced by two parallel plate transducers T1 and T2, each having front and back electrodes. In such embodiments, the electrical connections and operation of the plunger electrode and planar piezoelement electrodes will be similar to those employing a cylindrical transducer.

### Shear Wave Generation Using SP

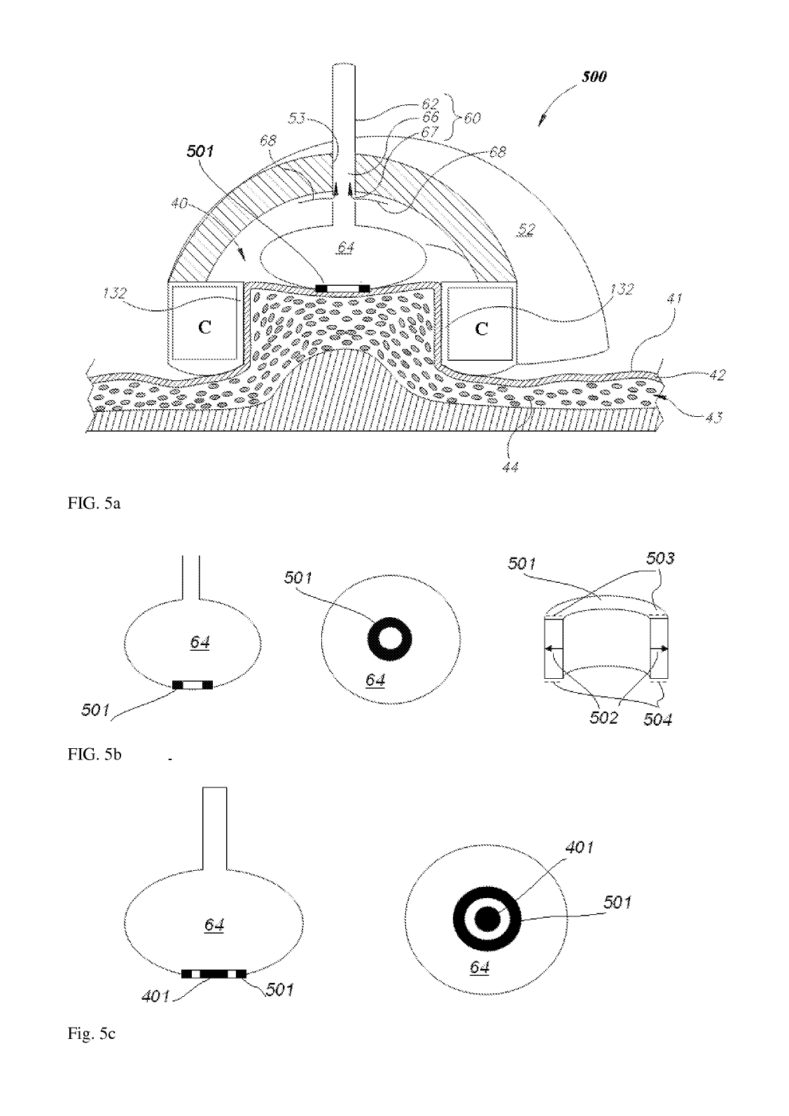

FIG. 5a shows schematically a cross section of a perspective view of an apparatus 500 according to another embodiment. Apparatus 500 includes a shear mode receiving (or just “shear mode”) element 501 attached to plunger head 64. More details of the arrangement are shown in FIG. 5b. In FIG. 5b, element 501 has a ring shape. Shear mode elements are known in the art and are usually made from piezoceramics with a polarization direction such as a direction 502 parallel to main surfaces covered by electrodes 503 and 504. A ring shape shear mode element has polarization along the radius of the ring. Its thickness must be equal to half a wavelength for working frequency, but the element can be also very thin (0.5 mm or thinner) and broadband (damped by a damper—not shown) for receiving of any shear wave (non-resonance receiver). Shear mode elements are used to “register” shear waves. The registration is needed to be able to get useful information on medium properties (e.g. to estimate shear wave velocity, attenuation, etc), i.e. for diagnostic purposes. FIG. 5c shows an embodiment in which RF electrode 401 and shear mode receiving element 501 are mounted on plunger head 64 for a type of combinational treatment described below. Note that in general, a shear mode element may be positioned differently within the apparatus and not necessarily attached to a plunger head.

The generation of shear waves 601 by an ultrasonic burst 202 is shown schematically in FIG. 6. Details may be found in PCT application PCT/IB2011/051917 by the present inventor.

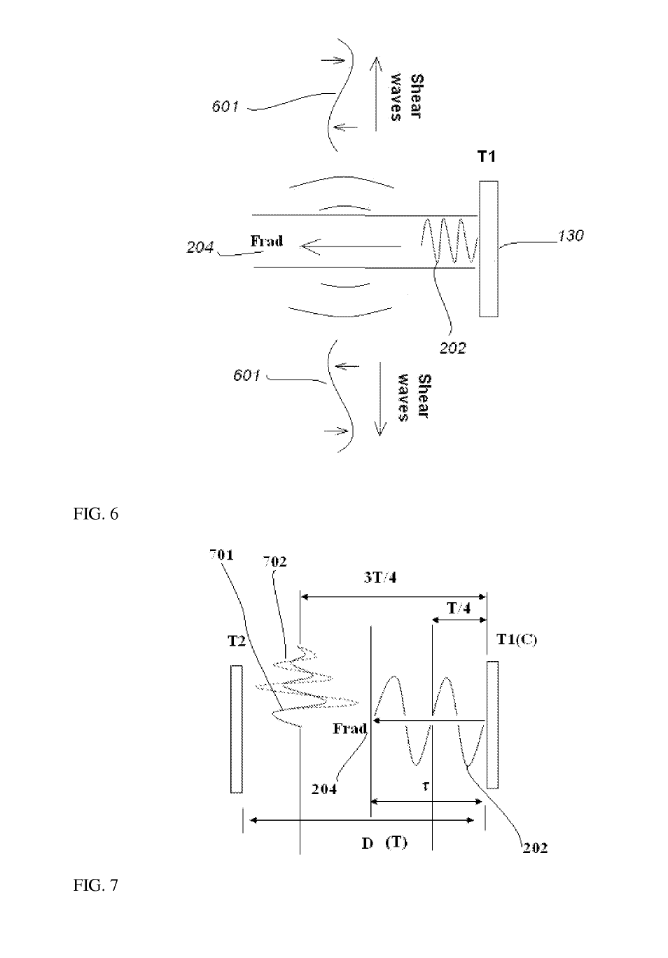

FIG. 7 shows schematically the generation of shear waves from the interaction of direct and reflected waves in a SP regime. Shear force F_{rad} generates shear deformation and, after the burst passes through a fixed plane, generates a shear wave which propagates in a lateral direction (perpendicular to the burst propagation direction, exemplarily up and down in the figure). For now, acoustic attenuation is ignored. After the first burst which generates a force F_{rad} with a first polarity is fully reflected at t1, as described above, the reflected burst with amplitude 2A generates a radiation force 4F_{rad} with a second, opposite polarity. The reflected burst also generates shear deformations and shear waves in the lateral direction. Thus, the ultrasonic burst runs forward and backward and generates and amplifies shear waves. For example, in the plane distanced from T1 (or along the diameter D of cylindrical piezoelement C) by 3T/4, an ultrasonic burst 202 generates a shear wave 701 propagating upwards. After reflection from T2 and amplification (doubling of amplitude) by a burst with opposite polarity, an upward shear wave 702 which is in phase with the second half period of shear wave 701 is generated, and its amplitude grows resonantly. The same situation appears in a plane distanced from T1 by T/4 (shear waves propagating down exist symmetrically but are not shown). In intermediate planes between T/4 and 3T/4, the shear waves are out of phase and compensate each other, but because the reflected bursts have doubled amplitude (double radiation force) additional attenuation shear waves are generated along the entire ultrasonic beam, their intensity being maximum in some planes and intermediate in all others. The intensity maximum positions can be changed by simply changing the burst length.

As well known, shear waves propagate slowly and attenuate strongly. For a 50 kHz shear wave (τ=10 μsec) the propagation velocity is Vₛₕₑₐᵣ=3 m/sec and the wavelength is λₛₕₑₐᵣ=0.06 mm. This means that for the time of a burst reflection in plane 3T/4 (t=10 μsec, burst velocity 1500 m/sec) shear waves propagate to a distance of 3 m/sec/50 kHz=0.06 mm from the center line of the transducers (i.e. to one shear wavelength). Taking into account the very high attenuation of shear waves (at 50-100 kHz, shear waves fully attenuate at a distance of 3-4 wavelengths), the method described herein can provide high intensity shear deformations localized in a narrow sub-skin tissue layer.

### EXAMPLE

If a burst length τ=10 μsec and the duty cycle is 1/4 (i.e. 10 μsec burst, 30 μsec pause), the generated shear waves have a period Θ=20 μsec, i.e. a frequency equal to 50 kHz=1/Θ (in each plane on the ultrasonic burst path). In the general case, a shear wave will have a period equal to Θ=2τ (if the radiation force creates shear deformation during a time equal to τ−Θ/2, then the radiation force is zero and the medium starts to vibrate from + to − deformation).

If the transit time T between T1 and T2 (or along diameter D of C) is given by T=D/V and if the burst has a length τ=T/2, the shear deformations generated by a reflected burst will be in phase with the shear wave generated by the direct burst in each plane for which n′τ/2=nT/4 where n is odd, i.e. in planes distanced (in time scale) from T1 (or along diameter D from the perimeter of C) by τ/2=T/4 and 3τ/2=3/4T.

For τ=T/3, shear deformations generated by the reflected burst will be in phase with the shear wave generated by the direct burst in each plane for which nτ/2=nT/6, where n is odd, i.e. in planes distanced (in time scale) from T1 (or along diameter D from the perimeter of C) by τ/2=T/6, 3τ/2=T/2 and 5τ/2=5T/6.

For τ=T/4, shear deformations generated by the reflected burst will be in phase with the shear wave generated by the direct burst in each plane for which nτ/2=nT/8, where n is odd, i.e. in planes distanced (in time scale) from T1 (or along diameter D from the perimeter of C) by τ/2=T/8, 3τ/2=3/8T, 5τ/2=5/8T and 7τ/2=7/8T. Similar expressions can be obtained for τ=T/m, where m>4.

In a particular numerical example, for a burst length T=10 μsec, T=20 μsec, D=30 mm and a longitudinal ultrasound velocity in tissue 1500 m/sec, one will get shear waves with period Θ=2τ=20 μsec i.e. frequency f=1/T=50 kHz, and positions of “in phase” shear waves will be τ/2=T/4=5 μsec and 3τ/2=3/4τ=15 μsec.

### Shear Wave Generation in the SP Regime by Multi-Electrode Piezoelements

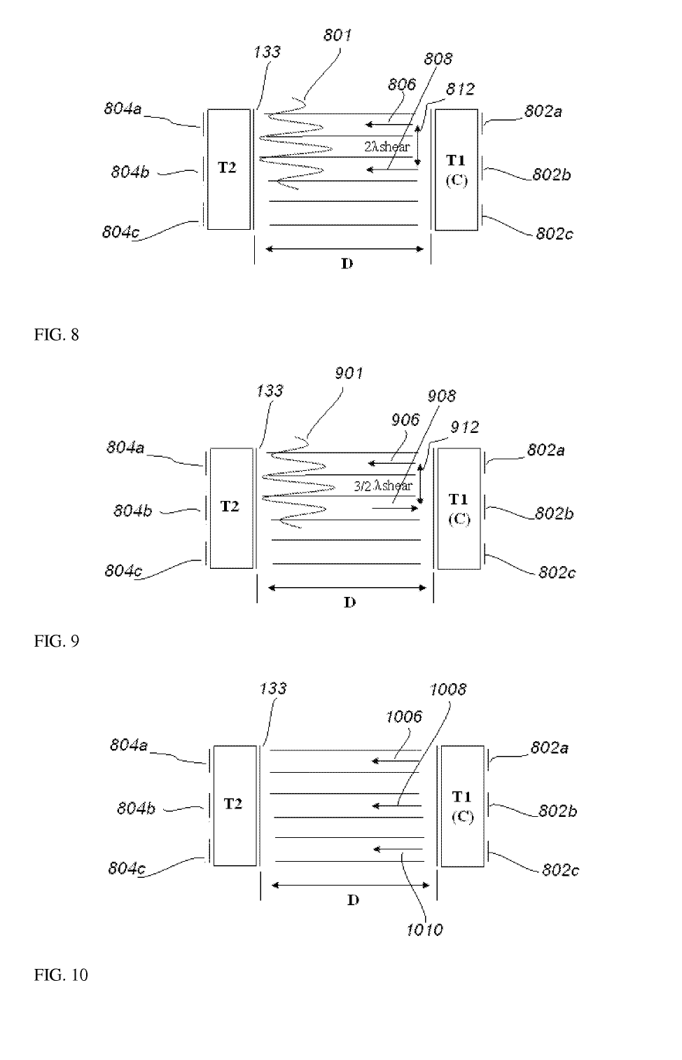

FIG. 8 shows schematically one use of an apparatus with multi-electrode transducers used to produce shear waves. The use is shown for parallel transducers T1 and T2. For a cylindrical transducer configuration the operation is the same, except that the multi-electrodes are placed as back electrodes on a cylindrical piezoelement. T1 (or C) has an array of back electrodes 802a, 802b and 802c, and T2 has an array of back electrodes 804a, 804b and 802c. Although only 3 electrodes are shown, there can be more. There is a single front electrode 133. Electrodes 802a on T1 and 804a on T2 are excited by a burst with τT=T/2 and generate an ultrasonic beam (burst) 806 in the SP regime as described above. This produces shear waves (like a shear wave 801) along the entire ultrasonic beam length D, and intensive shear waves in planes T/4 and 3T/4. If electrodes 802b and 804b are excited simultaneously with 802a and 804a to generate two parallel ultrasonic beams 806 and 808, and if the distance 812 between the centers of these beams equals nλₛₕₑₐᵣ/2 where n is even, then there is additional amplification of shear waves generated by the two beams. These two beams can be treated like virtual resonators of shear waves. Virtual resonators are described in detail in PCT application PCT/IB2011/051917.

FIG. 9 shows schematically another use of the apparatus with multi-electrode parallel transducers of FIG. 8 to produce shear waves. A shear wave 901 is generated by two ultrasonic beams 906 and 908 emitted simultaneously by 802a and 804b. Two ultrasonic beams may also be emitted simultaneously by 802b and 804a as well as by other pairs, leading to shear wave generation as above. In this embodiment, a distance 912 between the centers of beams 906 and 908 is fixed at nλₛₕₑₐᵣ/2 where n is odd, because of the opposite direction of the ultrasonic bursts (radiation forces) and, as a result, the opposite shear deformations. The beams are narrow and spaced by fixed distances (odd half-wavelength for opposite directions and even half-wavelength for parallel directions of bursts) for any number of electrodes.

### Shear Wave (SSW) Generation in a Supersonic Regime

FIG. 10 shows schematically a use of an apparatus with multi-electrode transducers to produce shear waves in a supersonic regime (also referred to herein as “supersonic shear waves” (SSWs)) or to produce shear wave resonance (SWR). In FIG. 10, the configuration of electrodes is the same as in FIGS. 8 and 9, but the electrode couples are now switched on successively instead of simultaneously, i.e. 802a-804a, then 802b-804b and then 802c-804c. This leads to successive generation of ultrasonic beams 1006, 1008 and 1010 and to the step by step move of a beam down (e.g. beam 1006) or up (e.g. beam 1010). At appropriate distances between beams and switching times (delays), one can get SWR or a SSW regime. Supersonic shear waves are described in detail in PCT/IB2010/052624. A supersonic regime is realized if a beam is moved in the shear wave propagation direction with a velocity higher than the shear wave velocity and when there is some constructive interference (similar to that of a Mach cone from an airplane).

Let the distances between ultrasonic beams 1006, 1008 and 1010 be equal to shear wavelength λₛₕₑₐᵣ (or to a multiple n of this wavelength, nλₛₕₑₐᵣ). Let electrodes 802a-804a be switched on at time t=0 to generate a burst of length τ=T/2=10 μsec and then work in a SP regime. Electrodes 802b-804b are then switched on at time t1=20 μsec and electrodes 802c-804c are switched on at time t2=40 μsec. Consequently, the SP ultrasonic beam position is moved from 1006 to 1008 to 1010 or vice versa. As a result, the shear waves generated by the first beam reach the center line of the next beam when the next beam is switched on (in phase) and amplify a shear wave resonantly. This effect is superposed on the synchronous pumping and resonant amplification of shear waves in definite planes described above. For SSW generation, an ultrasonic beam has to move faster than a shear wave generated by the same beam, i.e. switching times t1 and t2 must be less than or equal to t1=20 μsec and t2=40 μsec.

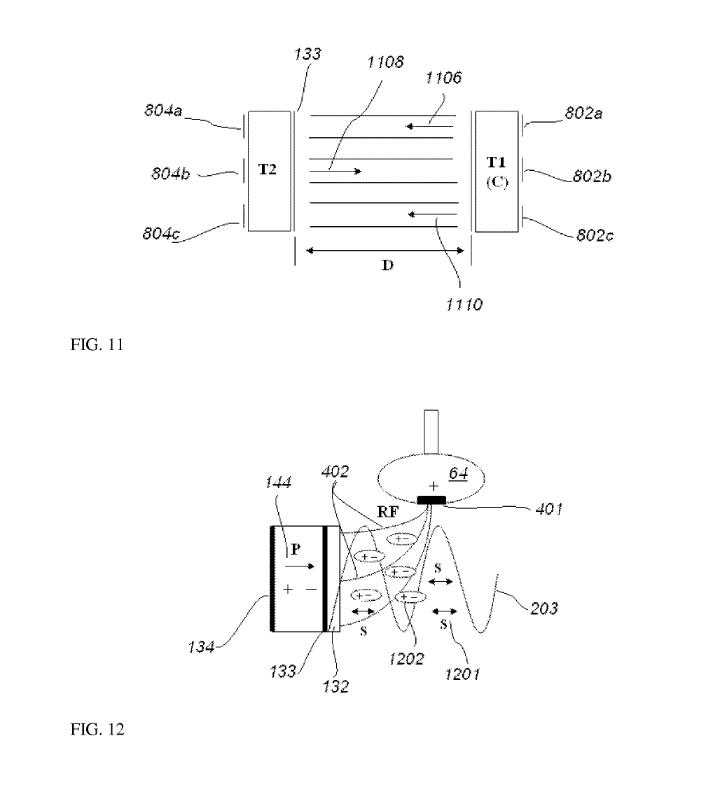

FIG. 11 shows schematically another use of an apparatus with multi-electrode transducers to produce supersonic shear waves. In this embodiment, electrodes arranged as in FIG. 9 are used to switch on beams 1106, 1108 and 1110 sequentially in the directions shown, and shift the beams up or down. The distances between ultrasonic beams 1006, 1008 and 1010 in this case must be equal to half of shear wavelength λₛₕₑₐᵣ/2 (or to a multiple n of half of wavelength nλₛₕₑₐᵣ/2, where n is an odd integer).

To realize a quasi-continuous movement of ultrasonic beams, the distances between beam centers for both cases shown in FIGS. 10 and 11 can be very small, on the order of 100 micrometers. The shifting “step” can be of the same order, while a beam width can be 2-3 mm. To realize such fine shifting, each back electrode can be subdivided into a plurality of very thin strips with width and gaps of tens of micrometers, as in a standard linear ultrasonic transducer array. Exemplarily, each back electrode may be divided into 20 such strips. In this case, the ultrasonic beam can be moved up or down with a fine step (“quasi-continuously”) at shear wave velocity (providing shear wave resonance (SWR) in a “wave resonance regime”) or at a higher than shear wave velocity (“SSW regime”) using appropriate switching times.

An apparatus as in FIGS. 5a, b may be used as a measurement cell for measuring tissue properties before and after treatment, for assessment of shear tissue properties (e.g. shear elastic modulus). Shear waves generated by any of the methods described in FIGS. 7-11) are received by the shear mode element. This enables to measure the shear wave velocity (transit time) and calculate the shear elastic modulus of the tissue (from a known distance from the SP beam to the shear mode element and a measured transit time for shear waves). Such a measurement is more informative than longitudinal elastic modulus measurements.

### Exemplary Shear Wave Applications

Shear waves generated by wave resonance can be used in various applications. As shown above, shear deformation created by a burst radiation force can be localized in a narrow sub-skin layer, namely in the dermis and epidermis layers where hair roots are placed. Therefore, shear deformations can act like a shaver. Other applications include cellulite removal, skin renewal and fat reduction by shear waves-assisted therapy (shear waves supplemented by ultrasonic burst treatment, etc.). The biological mechanism of shear wave influence on tissue and cells is described in PCT/IB2011/051917. Note that the shear wave frequency depends on burst length. Attenuation and wavelength of shear waves depends on frequency. Therefore, one can use shear wave frequencies at which a shear wave will attenuate practically on one wavelength, and this wavelength can be a tenth of a micron—close to a cell size (causing effective destruction of cells).

### Combinational Treatment Using SP, SWR, SSW and RF

The SP, SWR and SSW effects described above can be applied in a “combinational” treatment of tissue, by adding RF capabilities to each of the apparatuses described above and by ensuring that the burst compensation layers are electrically conductive. In addition, application of vacuum suction on tissue, as described in detail in PCT/IB2010/052624, can be used with any apparatus and any method described herein for various treatments. It is well known that RF frequencies of hundreds of KHz to MHz range are effective for RF thermal treatment of live tissues. Physically, the origin of tissue heating by RF is due to ion movements and vibrations as well as orientation and rotations of polar molecules (dipoles) like in dielectrics and electrolytes. In the low MHz range, RF dielectric losses in living tissue have minor importance. At 1-2 MHz, the electrical resistance of tissue with high electrolyte (water with dissolved salts) content, like blood, muscles and internal organs is equal to 100-200 Ohm·cm. In tissue with low electrolyte content, like fat and bones, the electrical resistance is much higher and equals 2000-5000 Ohm·cm. The thermal influence of RF is in direct relationship to the squared current density, which is usually 0, 01-0, 03 A/cm².

FIG. 12 shows schematically a physical mechanism of combinational treatment using SP, SWR, SSW and RF. An AC burst voltage (exemplarily at 100 kHz-2 MHz) is supplied to a piezoelement (e.g. in T1, T2 or C) to obtain SP, SWR and SSW regimes as explained above. RF electrode 401 mounted to plunger 64 is AC-electrically excited as described above in opposite polarity with a front electrode 133. Burst compensation layers 132 are made conductive (e.g. of conductive rubber), so the AC electric voltage synchronously appears in tissue as RF lines 402, leading to RF heating. The SP, SWR and SSW ultrasonic fields provide periodical vibrations of tissue together with ions (deformations S). The spatial distributions of the SP, SWR and SSW ultrasonic fields and the RF electric field coincide. Synchronous (at the same frequency) excitation of RF fields and ultrasonic waves (in form of SP) and simultaneous excitation of SWR and SSW at low frequency (as discussed above, the shear waves frequency is in low kHz range and as a result displacement amplitudes are very high) will lead to multi-resonant movements of ions (ions will vibrate under AC fields, RF and mechanical deformations S in ultrasonic fields). SP, SWR and SSW will intensify the ion movements, leading to current increase and additional RF heating.

Note that combinational treatment using SP, SWR, SSW and RF is non-trivial, because only in the transducer configurations disclosed herein do RF and ultrasonic fields (RF, SWR, SSW′ and SP) coincide spatially and are superimposed synchronously to provide new biological and physical effects.

In summary, in some embodiments, there is provided synchronous pumping of ultrasonic waves without standing wave formation but with all related advantages: safety (minimal ultrasound intensity on the skin), efficiency (resonance grows of ultrasonic burst amplitude) and capability for continuous treatment of a tissue region temporarily fixed by vacuum inside an ultrasound transducer without nodal structure inherent for standing waves. In some embodiments, there is provided shear wave generation in a SP regime. In some embodiments, there is provided shear wave resonance. In some embodiments, there are provided supersonic shear waves. In some embodiments, there are provided combinational treatments using a combination of SP ultrasonic waves +RF, SWR+RF, SP+SWR+RF, SP+SWR+SSW+RF, and each of these combinations plus vacuum massage.

In the description and claims of the present application, each of the verbs, “comprise” “include” and “have”, and conjugates thereof, are used to indicate that the object or objects of the verb are not necessarily an exhaustive listing of members, components, elements or parts of the subject or subjects of the verb.

Various embodiments described herein are provided by way of example and are not intended to limit the scope of the invention. The described embodiments comprise different features, not all of which are required in all embodiments. Some embodiments utilize only some of the features or possible combinations of the features. Variations of embodiments and embodiments comprising different combinations of features than those noted will occur to persons of the art. The scope of the invention is limited only by the following claims.

All patents, patent applications and publications mentioned in this specification are herein incorporated in their entirety by reference into the specification, to the same extent as if each individual patent, patent application or publication was specifically and individually indicated to be incorporated herein by reference. In addition, citation or identification of any reference in this application shall not be construed as an admission that such reference is available as prior art.

1-12. (canceled)

13. A method for producing resonantly amplified beams of ultrasonic bursts, comprising the steps of:
(a) providing a cylindrical piezoelement surrounding a medium;
(b) using the cylindrical piezoelement to emit a direct ultrasonic burst which has a respective amplitude and polarity and which propagates in the medium until it is reflected by the cylindrical piezoelement, thereby creating a reflected ultrasonic burst which has a polarity and propagation direction opposite to those of the direct ultrasonic burst; and
(c) in synchronization with the creation of the reflected ultrasonic burst, using the cylindrical piezoelement to emit a new direct ultrasonic burst, whereby the new emitted ultrasonic burst and the reflected ultrasonic burst combine to form an amplified ultrasonic burst which has an amplified amplitude and which propagates in the medium until it arrives at, and is reflected by the cylindrical piezoelement;
whereby repetition of the emission of a new direct ultrasonic burst in synchronization with the reflection of newly arrived ultrasonic burst creates in the medium synchronously pumped (SP) ultrasonic burst waves with increasing amplitudes.

14. The method of claim 13, wherein the emitted and reflected ultrasonic bursts have equal amplitudes.

15. (canceled)

16. The method of claim 13, wherein the medium includes biological tissue.

17. The method of claim 16, further comprising the step of:
(d) providing radio frequency (RF) energy to the biological tissue, thereby obtaining an amplified ultrasonic burst and RF effect in the medium.

18. The method of claim 13, further comprising the step of:
(d) using the SP ultrasonic bursts to create SP ultrasonic shear waves; and
(e) providing a shear mode element operative to receive and and register the SP ultrasonic shear waves for diagnostics of the medium.

19. The method of claim 13, wherein the cylindrical piezoelement includes a single front electrode and a plurality of back electrodes, the method further comprising the step of:
(d) switching the back electrodes to emit ultrasonic bursts which create synchronously pumped ultrasonic shear waves with wavelength λₛₕₑₐᵣ.

20. The method of claim 19, wherein the step of switching the back electrodes includes simultaneously emitting two parallel and adjacent ultrasonic beams which propagate in the same direction in the medium, the two ultrasonic beams having centers separated by nλₛₕₑₐᵣ/2 where n is an even integer.

21. The method of claim 19, wherein the step of switching the back electrodes includes simultaneously emitting two parallel and adjacent ultrasonic beams which propagate in opposite directions in the medium, the adjacent ultrasonic beams having centers separated by nλₛₕₑₐᵣ/2 where n is an odd integer.

22. The method of claim 13, wherein each piezoelement includes a single front electrode and a plurality of back electrodes, the method further comprising the step of:
(d) switching the back electrodes to emit ultrasonic bursts which create supersonic shear waves with wavelength λₛₕₑₐᵣ.

23. The method of claim 22, wherein the step of switching the back electrodes includes successively emitting parallel and adjacent ultrasonic beams which propagate in the same direction in the medium, the adjacent ultrasonic beams having centers separated by nλₛₕₑₐᵣ/2 where n is an even integer.

24. The method of claim 22, wherein the step of switching the back electrodes includes successively emitting two parallel and adjacent ultrasonic beams which propagate opposite directions in the medium, the adjacent ultrasonic beams having centers separated by nλₛₕₑₐᵣ/2 where n is an odd integer.

25. An apparatus for producing resonantly amplified beams of ultrasonic bursts in a medium, comprising:
(a) at least one piezoelement coupled to the medium and operative to emit and reflect ultrasonic bursts;
(b) a power supply configured to excite each piezoelement to emit a direct ultrasonic burst in synchronization with the reflection of a previously emitted ultrasonic burst arriving at the same piezoelement, whereby the direct ultrasonic burst and the reflected ultrasonic burst combine to form a resonantly amplified ultrasonic burst wave in a synchronously pumped (SP) regime.

26. (canceled)

27. The apparatus of claim 25, wherein the at least one piezoelement includes one cylindrical piezoelement.

28. (canceled)

29. (canceled)

30. The apparatus of claim 29, 27 further comprising:
(c) a vessel comprising the at least one piezoelement;
(d) a vacuum pump that generates vacuum in the vessel for drawing up a tissue region into the vessel; and
(e) a protruding element which protrudes into the vessel and distorts the drawn up tissue region to improve coupling of the region to the at least one piezoelement.

31. The apparatus of claim 25, wherein a resonantly amplified ultrasonic burst in a synchroneously pumped (SP) regime creates a SP ultrasonic shear wave and wherein the apparatus further comprises a shear mode element operative to receive and register the SP ultrasonic shear wave for diagnostics of the medium.

32. The apparatus of claim 31, wherein the shear mode element is physically attached to the protruding element.

33. (canceled)

34. (canceled)

35. (canceled)

36. (canceled)

37. The apparatus of claim 30, wherein the protruding element includes an electrode, wherein each piezoelement includes at least one front electrode and at least one back electrode and wherein the power supply is configured to create a radio frequency (RF) electric field between the at least one back electrode and the protruding element electrode, the RF electric field used to heat the tissue.

38. The apparatus of claim 37, wherein each back electrode is divided into a plurality of electrode strips, and wherein the power supply is further configured to switch the back electrode strips to emit ultrasonic bursts which create synchronously pumped ultrasonic shear waves with wavelength λₛₕₑₐᵣ.

39. The apparatus of claim 38, wherein the power supply is further configured to switch the back electrode strips to simultaneously emit two parallel and adjacent ultrasonic beams which propagate in the same direction in the tissue, the adjacent ultrasonic beams having centers separated by nλₛₕₑₐᵣ/2 where n is an even integer.

40. The apparatus of claim 38, wherein the power supply is further configured to switch the back electrode strips to simultaneously emit two parallel and adjacent ultrasonic beams which propagate in opposite directions in the tissue, the adjacent ultrasonic beams having centers separated by nλₛₕₑₐᵣ/2 where n is an odd integer.

41-46. (canceled)
